(19) United States
(12) Patent Application Publication
(10) Pub. No.: US 2024/0374649 A1
(43) Pub. Date: Nov. 14, 2024

(54) Method for Treating Ilk Signaling Pathway Related Diseases Using Exosome Derived from Mesenchymal Stem Cells, and Pharmaceutical Composition

(71) Applicants: TSINGTAO A-SMART MEDICAL TECHNOLOGY CO., LTD., Shandong (CN); SHANDONG UNIVERSITY, Shandong (CN)

(72) Inventors: Zijiang CHEN, Shandong (CN); Jinlong MA, Shandong (CN); Gang LU, Shandong (CN); Ruican CAO, Shandong (CN); Ling GENG, Shandong (CN); Hongbin LIU, Shandong (CN); Yueran ZHAO, Shandong (CN); Yue LV, Shandong (CN)

(21) Appl. No.: 18/691,547

(22) PCT Filed: Sep. 15, 2022
(86) PCT No.: PCT/CN2022/118951

§ 371 Date: Mar. 13, 2024

(30) Foreign Application Priority Data
Sep. 15, 2021 (CN) 202111078183.5

(51) Int. Cl. A61K 35/28; A61P 15/08; C12N 5/0775

(52) U.S. Cl. CPC A61K 35/28; A61P 15/08; C12N 5/0662; C12N 2506/1307

(57) ABSTRACT

A method for regulating an ILK signaling pathway in a cell, includes administering to the cell exosomes derived from mesenchymal stem cells, in particular mesenchymal stem cells derived from induced pluripotent stem cells. A method is provided for treating ILK signaling pathway related diseases using the exosomes. A pharmaceutical composition is provided for treating ILK signaling pathway related diseases, and includes exosomes derived from mesenchymal stem cells.

This application claims the priority of the Chinese patent application 202111078183.5 with the title of “Method and Pharmaceutical Composition for Treating ILK Signaling Pathway Related Diseases with Exosomes Derived from Mesenchymal Stem Cells” filed on Sep. 15, 2021, the entire contents of which are incorporated in this application.

### TECHNICAL FIELD

The present invention relates to the field of cytology and pharmacology. Specifically, the present invention provides a method for treating diseases related to ILK signaling pathway using exosomes derived from mesenchymal stem cells and a pharmaceutical composition for treating diseases related to ILK signaling pathway.

### BACKGROUND

Integrin-linked kinase (ILK, also known as p59ILK) was originally identified by its ability to bind and phosphorylate the B1-integrin cytoplasmic domain from a two-hybrid screen of a human placental cDNA library (Hannigan et al., Nature, 1996). It was found that overexpression of ILK can lead to the destruction of epithelial morphology of IEC18 cells and the decrease of cell adhesion to extracellular matrix. Studies have also found a role for ILK in activating transcription in the Wnt signaling cascade (Novak et al., Proc. Natl. Acad. Sci. USA, 1998). The activity of ILK has been shown to be regulated in other signaling pathways, including those involving G proteins (Tu et al., Mol. Cell. Biol., 1999), phosphatidylinositol 3-kinase, protein kinase B and glycogen synthase Signaling pathway of kinase 3 (Delcommenne et al., Proc. Natl. Acad. Sci. U.S.A., 1998). It is also known in the art that the ILK signaling pathway is associated with angiogenesis, which has important implications for physiological and pathological angiogenesis. Endothelial ILK plays a critical role in vascular development and endothelial cell (EC) survival through integrin-matrix interactions.

Mesenchymal stem cells are important members of the stem cell family. Natural mesenchymal stem cells originate from the early developmental mesoderm. Mesenchymal stem cells can be isolated and cultured from various tissues. Mesenchymal stem cells can also be differentiated from pluripotent stem cells. Among them, induced pluripotent stem cells (induced pluripotent stem cells, iPSCs), also known as induced pluripotent stem cells or artificial pluripotent stem cells, are artificially prepared cells with the stemness of embryonic stem cells. Induced pluripotent stem cells were successfully prepared for the first time in 2006 by Japanese scientist Shinya Yamanaka, using viral vectors to transfer a combination of four transcription factors (Oct4, Sox2, Klf4 and c-Myc) into differentiated somatic cells, so that A cell type similar to embryonic stem cells and embryonic APSC pluripotent cells obtained by its reprogramming. Induced pluripotent stem cells have been widely used in the fields of biotechnology and medical research.

Recent discoveries surrounding the function of extracellular vesicles (EVs, small particles 40-100 nm in size) secreted by stem cells have led to major advances in regenerative medicine. The regenerative effect of stem cell transplant therapy is partly due to the paracrine effect of EV release. EVs do not contain MHC I or MHC II proteins, do not increase the risk of immunogenicity, and are not tumorigenic, thus overcoming several disadvantages of cell transplantation therapy. Extracellular vesicles, or exosomes, derived from mesenchymal stem cells (MSC-EVs) have been found to help repair injured tissues (Harrell et al., 2019; Tang et al., 2021). Human induced pluripotent stem cell-derived mesenchymal stem cells (iPSC-MSCs) have also been used in regenerative medicine. EVs secreted by iPSC-MSCs promote wound repair (Zhang et al., 2015), angiogenesis (Hu et al., 2015), bone angiogenesis (Hu et al., 2015) and bone regeneration (Qi et al., 2016). It has been found that EVs derived from mesenchymal stem cells can transfer functional miRNAs to target cells to regulate their functions, resulting in therapeutic effects in damaged tissues. For example, miR-644-5p in stem cells improved ovarian function in rats with chemotherapy-induced ovarian injury by targeting P53 (Sun et al., 2019). Similarly, EVs secreted by human amniotic mesenchymal stem cells release miRNA-320a, which regulates SIRT4 to protect POI mice from ovarian oxidative stress (Ding et al., 2020a). miRNA-17-5p derived from human umbilical cord mesenchymal stem cells improves ovarian function after chemotherapy by regulating SIRT7 (Ding et al., 2020b). Exosomes produced by bone marrow mesenchymal stem cells can target PTEN by delivering mir-144-5p, thereby improving ovarian function in rats with chemotherapy-induced ovarian insufficiency (Yang et al., 2020). However, these studies also found that the function in target cells, their mechanism of action, and the involved pathway members (proteins, genes or their regulators, etc.) of stem cells from different sources and the EVs they secrete are significantly different.

This field needs to study the influence of exosomes from various kinds of stem cells or other cellular sources, including the influence thereof on integrin-linked kinase, that is, ILK pathway, and the role in the treatment of diseases related to ILK signaling pathway, thus providing new effective and safe therapies and drugs for the treatment of diseases related to ILK signaling pathway.

### SUMMARY

This application provides a new therapy and pharmaceutical composition for treating ILK-related diseases using exosomes derived from mesenchymal stem cells. The inventors of the applicant discovered for the first time that exosomes derived from mesenchymal stem cells can regulate the expression of related proteins in the ILK signaling pathway of cells, including ILK, and can be used to treat ILK-related diseases.

In one aspect of the present invention, a method for regulating ILK signaling pathway in a cell is provided, which includes applying exosomes derived from mesenchymal stem cells to the cell or cells in tissue or culture medium.

In the present invention, the term mesenchymal stem cell is also called multipotent mesenchymal cells, mainly obtained from fat or bone marrow, capable of differentiating into various cells of mesoderm origin, such as bone, fat, cartilage, tendon and muscle, etc. Mesenchymal stem cells can be isolated and cultured from various tissues, but their abilities and cell surface markers vary according to their source. Mesenchymal stem cells are generally defined by cells that can differentiate into bone cells, chondrocytes, and muscle cells, and express cell surface markers such as CD73(+), CD105(+), CD34(−), and CD45(−).

In one aspect of the present invention, the mesenchymal stem cells are bone marrow-derived, adipose-derived, umbilical cord blood-derived, tooth-derived or pluripotent stem cell-derived mesenchymal stem cells. In yet another aspect of the present invention, the mesenchymal stem cells are derived from pluripotent stem cells.

In the present invention, the term pluripotent stem cells refers to stem cells capable of giving rise to all embryonic cell types. Natural pluripotent stem cells include embryonic stem cells. Induced pluripotent stem cells (iPSCs), also known as artificial pluripotent stem cells, are artificially prepared cells with the stemness of embryonic stem cells. For example, four transcription factors (Oct4, Sox2, Klf4 and c-Myc) are transferred into differentiated somatic cells and reprogrammed.

In one aspect of the present invention, the mesenchymal stem cells are human induced pluripotent stem cell-derived mesenchymal stem cells (hiPSC-MSC).

Extracellular vesicles are membrane vesicles secreted by cells. Extracellular vesicles may have a diameter (in the case of a particle other than a sphere, its largest dimension) of between about 10 nm and about 5000 nm. In the present invention, exosomes refer to small secretory vesicles, typically having a diameter (in the case of a particle other than a sphere, its largest dimension) of between about 30 nm and about 250 nm, for example about 30 nm to about 250 nm. diameter between about 200 nm. Exosomes contain nucleic acids, proteins, or other biomolecules or have nucleic acids, proteins, or other biomolecules in their membranes and can act as carriers between different locations in the body or biological systems.

Exosomes can be isolated from a variety of biological sources including mammals such as mice, rats, guinea pigs, rabbits, dogs, cats, cows, horses, goats, sheep, primates or humans. Exosomes can be isolated from biological fluids such as serum, plasma, whole blood, urine, saliva, breast milk, tears, sweat, joint fluid, cerebrospinal fluid, semen, vaginal fluid, ascitic fluid, and amniotic fluid. Exosomes can also be isolated from experimental samples such as culture medium taken from cultured cells.

In one aspect of the present invention, a method for regulating the ILK signaling pathway in cultured cells is provided, which comprises adding exosomes from induced pluripotent stem cell-derived mesenchymal stem cells to the cell culture medium. In one aspect of the present invention, the mesenchymal stem cells are cells that have undergone 1-15 passages, preferably have undergone 1-10 passages, and most preferably have undergone 3-7 passages.

In one aspect of the present invention, the amount of exosomes added to the culture medium is about 1-500 μg/ml, preferably about 5-250 μg/ml, more preferably about 10-200 μg/ml.

In one aspect of the present invention, the cells are cells with abnormal (down-regulated) ILK pathway (such as PTEN/ILK/AKT pathway). In yet another aspect of the present invention, in the method, the ILK pathway activity of the cell is restored, for example, the ILK activity of the cell is upregulated.

In one aspect of the present invention, the method includes the step of detecting ILK pathway (such as PTEN/ILK/AKT pathway) related genes, such as Ilk, Pten, Krt18, Ccnd1, Cdkn2a, Vegfa, Ptgs2.

In one aspect of the present invention, a method for treating diseases related to ILK signaling pathway is provided, which includes administering exosomes derived from mesenchymal stem cells to patients. In one aspect of the present invention, use of exosomes derived from mesenchymal stem cells for the preparation of medicaments for treating ILK-related diseases is also provided.

In one aspect of the present invention, a pharmaceutical composition for treating ILK-related diseases is provided, which contains exosomes derived from mesenchymal stem cells.

In one aspect of the present invention, use of exosomes derived from mesenchymal stem cells in a pharmaceutical composition for treating ILK-related diseases is provided.

In the present application, ILK-associated diseases are also referred to as ILK signaling pathway-associated diseases, which include diseases or disorders associated with changes in ILK expression and/or activity, including diseases or disorders responsive to modulation of ILK expression. The role of ILK in the activation of transcription in the Wnt signaling cascade is known in the art. ILK is also known to play a role in other signaling pathways, including those involving G proteins, phosphatidylinositol 3-kinase, protein kinase B, and glycogen synthase kinase 3. The upstream regulatory signals of ILK include, for example, PTEN (a lipid phosphatase that negatively regulates ILK activation) and so on. The known ILK signaling pathway also includes the downstream AKT pathway and so on. In one aspect of the invention, the ILK signaling pathway comprises the PTEN/ILK/AKT pathway.

It is known in the art that the ILK signaling pathway is related to angiogenesis, which is of great significance to physiological and pathological angiogenesis. Endothelial ILK plays a critical role in vascular development and endothelial cell (EC) survival through integrin-matrix interactions. Integrin-mediated signaling cooperates with vascular endothelial growth factor (VEGF) receptors to promote morphological changes, cell proliferation, and motility of endothelial cells. Thus, diseases associated with ILK signaling pathway include diseases associated with abnormal or pathological angiogenesis, such as, but not limited to, various cancers, psoriasis, and age-related macular degeneration.

Diseases associated with abnormal or pathological angiogenesis include cancers such as brain, esophagus, bladder, cervix, breast, lung, prostate, colorectal, pancreas, head and neck, prostate, thyroid, kidney carcinoma and ovarian cancer, melanoma, lymphoma, glioma, glioblastoma and any other cancers etc. In this regard, ILK signaling pathway-associated diseases also include heart diseases (e.g., cardiomyopathy, cardiovascular disease, congenital heart disease, coronary heart disease, heart failure, hypertensive heart disease, inflammatory heart disease, valvular heart disease).

ILK signaling pathway-related diseases also include a variety of known metabolic disorders including genetic metabolic disorders. The metabolic disorders include, but are not limited to, diabetes, hyperlipidemia, lactic acidosis, phenylketonuria, tyrosinemia, urea cycle disorders, and the like.

In addition, it is known in the art that the ILK signaling pathway is closely related to inflammation: leukocyte extravasation is an important step in inflammation, and integrins have been shown to play an important role by mediating the interaction of leukocytes with vascular endothelium and subendothelial extracellular matrix. As a link between integrins and the cytoskeletal system, ILK is a key molecule involved in cell-cell and cell-matrix interactions.

In this regard, diseases associated with ILK signaling pathway include various inflammatory diseases such as but not limited to asthma, chronic obstructive pulmonary disease, inflammatory bowel disease, ankylosing spondylitis, Reiter syndrome, Crohn's disease, ulcerative colitis, systemic lupus erythematosus, psoriasis, atherosclerosis, rheumatoid arthritis, osteoarthritis, or multiple sclerosis.

Studies have found that the AKT pathway downstream of ILK is the key to folliculogenesis, and disruption of the AKT pathway will impair the survival of primordial follicles and lead to POI (Kalich-Philosoph et al., 2013; Wang et al., 2019). In this regard, diseases related to ILK signaling pathway also include ovarian-related reproductive disorders, such as decreased ovarian function caused by chemotherapy, decreased ovarian reserve (DOR), premature ovarian insufficiency (POI), premature ovarian failure (POF), etc. Especially said diseases related to ILK signaling pathway is premature ovarian failure (POF).

In one aspect of the present invention, the mesenchymal stem cells used for generating exosomes in the aforementioned methods and pharmaceutical compositions are mesenchymal stem cells obtained after subculture of mesenchymal stem cells differentiated from induced pluripotent stem cells and the like. For example, the mesenchymal stem cells are cells that have passed 1-15 passages, preferably cells that have passed 1-10 passages, and most preferably cells that have passed 3-7 passages. In the present invention, the primary cells used for subculture, that is, P0 cells, usually refer to the mesenchymal stem cells that appear first after the mesenchymal stem cell-derived cells are induced and differentiated, that is, cells with mesenchymal stem cell characteristics (for example, having mesenchymal stem cell-specific surface markers, etc.) account for more than 50% of the total number of cells in the cell population, or preferably more than 75%, or more preferably more than 90%. Primary cells can be directly used for subculture, or can be used for subculture after recovery from cryopreservation. The inventors of the present application unexpectedly found that exosomes extracted from mesenchymal stem cells derived from induced pluripotent stem cells have a positive restorative effect on cells of damaged tissues (such as oocytes and granulosa cells of various stages in the ovary, etc.). The inventors of the present application further found that among the sub-cultured mesenchymal stem cells, the mesenchymal stem cells of early generations (such as the cells have undergone 1-15 passages or less, especially the cells have undergone 7 passages or less) generate exosomes which have significantly better repairing activity on damaged tissue cells (such as oocytes and granulosa cells in the ovary) than the exosomes generated by the late generations of mesenchymal stem cells (such as the cells have undergone 10-15 passages or more passages). At the same time, the inventors of the present application unexpectedly found that, compared to mesenchymal stem cells isolated from tissues (such as bone, fat, cartilage, umbilical cord), the mesenchymal stem cells formed by differentiation from induced pluripotent stem cells have a much better chance to generate exosomes with improved activities for repairing damaged tissue: one manifestation is that the variation in the quantity and the level of reparative activity on cells of damaged tissues of exosomes in different production lots generated from induced pluripotent stem cell-derived mesenchymal stem cells that have undergone 15 passages or less can be maintained within 20%; on the other hand, the variation in the quantity and the level of reparative activity on cells of damaged tissues of exosomes in different production lots generated from mesenchymal stem cells derived from fat cells or umbilical cord that have undergone around 10 passages is about 50% or higher.

Exosomes used in the present invention can be separated and/or purified from cell cultures by various methods known in the art. Methods for isolating exosomes include ultrafiltration, polymer precipitation, size chromatography, or ultracentrifugation. Preferably, the exosomes used in the present invention can be prepared by ultrafiltration.

In one aspect of the present invention, the exosomes used in the present invention are prepared from cell culture fluid by ultrafiltration. In the ultrafiltration method used in the present invention, exosomes are filtered using an ultrafiltration membrane with a molecular weight cutoff of about 100 kDa. Exosomes are present in fractions that fail to pass through ultrafiltration membranes with a molecular weight cut-off of approximately 100 kDa.

In the ultrafiltration method used in the present invention, a multi-filtration system and method can be used, that is, to filter through filters with different pore sizes. In yet another aspect of the present invention, the ultrafiltration method further includes a step of filtering with a filter with a pore size of 4 μm and/or a filter with a pore size of 0.22 μm before the ultrafiltration membrane with a molecular weight cut-off of about 100 kDa. For example, filtration can be performed stepwise through a cell filter with a pore size of 4 μm, a cell filter with a pore size of 0.22 μm, and a filter with a MWCO (molecular weight cut-off) of 100 kD.

The pharmaceutical composition for preventing or treating ILK signaling pathway-related diseases provided by the present invention contains a pharmaceutically effective amount of the afore-mentioned exosomes. The exosomes may be included in the pharmaceutical composition alone or together with one or more pharmaceutically acceptable carriers, excipients or diluents. A pharmaceutically effective amount means an amount sufficient to prevent, improve or treat the symptoms of diseases related to ILK signaling pathway.

In one aspect of the present invention, the dosage of the exosomes in the pharmaceutical composition provided by the present invention is about 1-500 μg, preferably about 5-250 μg, more preferably about 10-200 μg. In yet another aspect of the present invention, the dosage of the exosomes is about 40-5000 μg/kg body weight, preferably about 400-4000 μg/kg body weight.

In one aspect of the present invention, the dosage of the exosomes in the pharmaceutical composition provided by the present invention is about 1×10⁹ to 1×10¹² exosomes, preferably about 1×10¹⁰ to 1×10¹¹ exosomes. In yet another aspect of the present invention, the dosage of the exosomes is about 1×10¹⁰ to 4×10¹³ exosomes/kg body weight, preferably about 1×10¹¹ to 4×10¹² exosomes/kg body weight.

In addition, the pharmaceutical composition provided by the present invention can be prepared as a unit dose formulation suitable for administration to patients according to the usual methods in the field of pharmacy, and the formulation contains an effective administration amount of the afore-mentioned exosomes for one or several administrations. The pharmaceutical composition provided by the invention can be in a dosage form of single or multiple administrations. The pharmaceutically effective amount can be appropriately changed according to the severity of the disease, age, weight, health and sex of the patient, administration route, treatment period.

The exosomes used in the pharmaceutical composition provided by the present invention are particularly suitable for multiple administrations for treatment. In one aspect, the pharmaceutical composition provided by the invention is a dosage form for multiple administrations. In yet another aspect of the present invention, the pharmaceutical composition provided by the present invention is a dosage form administered at an interval of about 1 day to 7 days. Preferably, the pharmaceutical composition is in a dosage form administered at an interval of about 2-7 days.

In one aspect, in the dosage form of the pharmaceutical composition provided by the present invention, especially in the dosage form of multiple administrations, the dosage of each administration of the exosomes is about 1-500 μg, preferably about 5-250 μg, more preferably about 10-200 μg. In another aspect of the present invention, in the dosage form of the pharmaceutical composition provided by the present invention, especially in the dosage form of multiple administrations, the dosage of each administration of the exosomes is about 40-5000 μg exosomes/kg body weight, preferably about 400-4000 μg exosomes/kg body weight.

In one aspect, in the dosage form of the pharmaceutical composition provided by the present invention, especially in the dosage form of multiple administrations, the dosage of each administration of the exosomes is about 1×10⁹ to 1×10¹² exosomes, preferably about 1×10¹⁰ to 1×10¹¹ exosomes. In another aspect of the present invention, in the dosage form of the pharmaceutical composition provided by the present invention, especially in the dosage form of multiple administrations, the dosage of each administration of the exosomes is about 1×10¹⁰ to 4×10¹³ exosomes/kg body weight, preferably about 1×10¹¹ to 4×10¹² exosomes/kg body weight.

The pharmaceutical compositions provided herein are physiologically acceptable and generally do not cause allergic reactions, such as gastroenteropathy or dizziness or the like, when administered to humans. Examples of carriers, excipients and diluents may include lactose, dextrose, sucrose, sorbitol, mannitol, xylitol, erythritol, maltitol, starch, gum arabic, alginate, gelatin, Calcium phosphate, calcium silicate, cellulose, methylcellulose, polyvinylpyrrolidone, water, methylparaben, propylparaben, talc, magnesium stearate and mineral oil. In addition, fillers, deflocculants, lubricants, humectants, flavorants, emulsifiers, preservatives, etc. may also be included.

In addition to the active ingredient, the pharmaceutical preparation can also contain one or more pharmaceutically acceptable common inert carriers, such as preservatives, analgesics, solubilizers, stabilizers, etc. for injection or bases, excipients, etc. for surface preparations agents, lubricants or preservatives, etc.

Compositions or pharmaceutical formulations of the present disclosure prepared as described above can be administered to mammals, such as rats, mice, livestock, humans, etc., by various routes including parenteral and oral routes. Any mode of administration commonly used in the art can be used.

The present invention also provides a method for treating ILK-related diseases, which comprises administering exosomes derived from mesenchymal stem cells to patients. The patient can be a mammal such as rat, mouse, livestock, human, and the like. Any mode of administration commonly used in the art can be used.

In one aspect of the present invention, the mesenchymal stem cells used in the method for treating ILK-related diseases are bone marrow-derived, adipose-derived, umbilical cord blood-derived, tooth-derived or pluripotent stem cell-derived mesenchymal stem cells. In one aspect of the present invention, the mesenchymal stem cells used in the method for treating diseases related to ILK signaling pathway are mesenchymal stem cells derived from induced pluripotent stem cells.

In one aspect of the present invention, the mesenchymal stem cells used in the method for treating ILK-related diseases are cells that have undergone 1-10 passages, preferably cells that have undergone 3-7 passages.

In one aspect of the present invention, in the method for treating ILK-related diseases, the exosomes are prepared by ultrafiltration. In one embodiment, in the ultrafiltration method, an ultrafiltration membrane with a molecular weight cut-off of about 100 kDa is used to screen the exosomes. Preferably, the ultrafiltration method further includes the step of filtering with a filter with a pore size of 4 μm and/or a filter with a pore size of 0.22 μm before the ultrafiltration membrane with a molecular weight cut-off of about 100 kDa.

In one aspect of the present invention, in the method for treating ILK-related diseases, about 1-500 μg, preferably about 5-250 μg, more preferably about 10-200 μg of exosomes are administered to the patient. In yet another aspect of the present invention, in the method for treating diseases related to ILK signaling pathway, 40-5000 μg/kg body weight, preferably about 400-4000 μg/kg body weight of exosomes is administered to the patient.

In one aspect of the present invention, in the method for treating ILK-related diseases, about 1×10⁹ to 1×10¹² exosomes, preferably about 1×10¹⁰ to 1×10¹¹ exosomes are administered to the patient. In yet another aspect of the present invention, in the method for treating diseases related to ILK signaling pathway, about 1×10¹⁰ to 4×10¹³ exosomes/kg body weight, preferably about 1×10¹¹ to 4×10¹² exosomes/kg body weight, is administered to the patient.

In one aspect of the present invention, in the method for treating ILK-related diseases, the exosomes are administered to the patient one or more times.

In one aspect of the present invention, in the method for treating ILK-related diseases, the exosomes are administered to the patient at intervals of about 1-7 days, preferably, at intervals of about 2-7 days.

In one aspect of the present invention, in the method for treating ILK-related diseases, especially in the treatment method for multiple administrations, the dosage of each administration of the exosomes is about 1-500 μg, preferably It is about 5-250 μg, more preferably about 10-200 μg. In another aspect of the present invention, in the method for treating diseases related to ILK signaling pathway provided by the present invention, especially in the treatment method of multiple administrations, the dosage of each administration of the exosomes is about 40-5000 μg exosomes/kg body weight, preferably about 400-4000 μg exosomes/kg body weight.

In one aspect of the present invention, in the method for treating ILK-related diseases, the dosage of each exosome is about 1×10⁹ to 1×10¹² exosomes, preferably about 1×10¹⁰ to 1×10¹¹ exosomes. In another aspect of the present invention, in the method for treating diseases related to ILK signaling pathway provided by the present invention, especially in the treatment method of multiple administrations, each administration dose is about 1×10¹⁰ to 4×10¹³ exosomes/kg body weight, preferably about 1×10¹¹ to 4×10¹² exosomes/kg body weight.

In one aspect of the present invention, the method for treating an ILK-related disease is a method for treating a disease related to abnormal or pathological angiogenesis, such as cancer, heart disease, metabolic disorder, inflammatory disease or ovarian-related reproductive disorder.

### BRIEF DESCRIPTION OF THE DRAWINGS

FIG. 1, including FIGS. 1A to 1C, shows the results of identification of exosomes (iPSC-MSCs-EVs) obtained from human induced pluripotent stem cell-derived mesenchymal stem cells (iPSC-MSCs). The iPSC-MSCs-EVs were characterized by immunoblotting (FIG. 1A), transmission electron microscopy (FIG. 1B) and RNA distribution analysis (FIG. 1C), respectively.

FIG. 2, including FIGS. 2A to 2E, shows that iPSC-MSCs-EVs regulate ILK signaling pathway in granulosa cells of chemotherapy injury model.

FIG. 2A shows the RNA-sequencing differential gene volcano plots.

FIG. 2B shows a graph showing the results of the IPA software analysis.

FIG. 2C shows the difference in gene changes analyzed by Qlucore software.

FIG. 2D is the heat map analysis of differential genes related to ILK signaling pathway.

FIG. 2E is the analysis of qPCR results of differentially expressed genes related to ILK signaling pathway.

FIG. 3, including FIGS. 3A to 3C, shows that iPSC-MSCs-EVs can reverse the downregulation of PTEN/ILK/AKT pathway induced by CTX in vivo or in vitro experiments.

FIG. 3A is a graph showing the results of detecting the expression of ILK signaling pathway-related proteins in granulosa cells by immunoblotting.

FIG. 3B is a graph showing the results of detecting the expression of ILK signaling pathway-related proteins in in vitro cultured ovaries by immunoblotting.

FIG. 3C is a graph showing the results of immunohistochemical detection of ILK protein expression in in vitro cultured ovaries and adult mouse ovaries.

FIG. 4, including FIGS. 4A and 4B, is a graph showing the results of iPSC-MSCs-EVs treating CTX-induced apoptotic granulosa cells.

FIG. 4A is a diagram of the detection results of MTS.

FIG. 4B is a graph showing the results of detecting the expression of apoptosis markers in granulosa cells by immunoblotting.

FIG. 5, including FIGS. 5A and 5B, is a graph showing the effect of co-culture of iPSC-MSCs-EVs and 4HC-CTX-treated ovaries.

FIG. 5A is a graph of follicle counting results.

FIG. 5B is a diagram of the results of immunohistochemical assays.

FIG. 6, including FIGS. 6A to 6C, is a graph showing the effect on the ovaries of mice treated with CTX and those co-cultured with iPSC-MSCs-EVs.

FIG. 6A is a graph showing the results of HE staining experiments.

FIG. 6B is a graph of follicle counting results.

FIG. 6C is a graph showing the experimental results of detecting apoptosis-or proliferation-related proteins by immunohistochemistry.

### EXAMPLES

The invention is further illustrated by the following examples, but these examples do not limit the invention in any respect.

Example 1 Isolation and identification of exosomes from mesenchymal stem cells derived from human induced pluripotent stem cells.

Induction of iPSCs (Induced Pluripotent Stem Cells)

Mix 10⁶ human dermal fibroblasts (Human Dermal fibroblasts, ATCC #PCS-201-010) and 3 micrograms of plasmids (UL (addgene #27080), OP (addgene #27077), SK (addgene #27078)) in a solution. Co-electroporate the plasmids into the cells. Then the electroporated cells were planted on Matrigel-coated plates, cultured with mTeSR (STEMCELL #85850) for 24-29 days. Stain with alkaline phosphatase (AP) to evaluate the stemness of the cells to determine whether the somatic cells have been reprogrammed to be pluripotent stem cells.

Induction of iPSC-MSCs (Induced Pluripotent Stem Cell-Derived Mesenchymal Stem Cells)

According to the instructions of the kit, the human iPSCs obtained in the previous step were induced to be human induced pluripotent stem cell-derived mesenchymal stem cells (iPSC-MSCs) using the Mesenchymal Progenitor Kit.

Isolation of Exosomes from iPSC-MSCs

iPSC-MSCs are sub-cultured. Supernatants of iPSC-MSCs which have been sub-cultured for 3 times to those for 7 times (cells having undergone 3-7 passages) were collected, and exosomes (EVs) were isolated therefrom using ultrafiltration (Milipore, 100 kDa).

Experimental materials: refrigerated centrifuge (Eppendorf 5804R), Amicon® Ultra-15 Centrifugal Filter Unit (Millipore, 100KD #UFC910096).

The main steps of ultrafiltration:
1) Centrifuge the collected cell supernatant at 300 g, 4° C. for 10 min to remove residual cells;
2) Centrifuge at 2000 g for 20 min to remove cell debris;
3) Collect the supernatant and filter it with a 0.22 μm pore size filter;
4) Add the collected supernatant into Amicon® Ultra-15, centrifuge at 3000 g;
5) After the ultrafiltration of the supernatant is completed, add PBS and perform ultrafiltration again, repeat the washing twice, and finally dissolve the exosomes remaining on the filter membrane with 200 μl PBS to obtain an exosome solution.

The identification and observation of exosomes produced by MSCs include the use of electron microscopy and the observation of EV morphology and size, and the identification of the expression of EV surface markers CD9, CD63, and CD81 using Western blot technology.

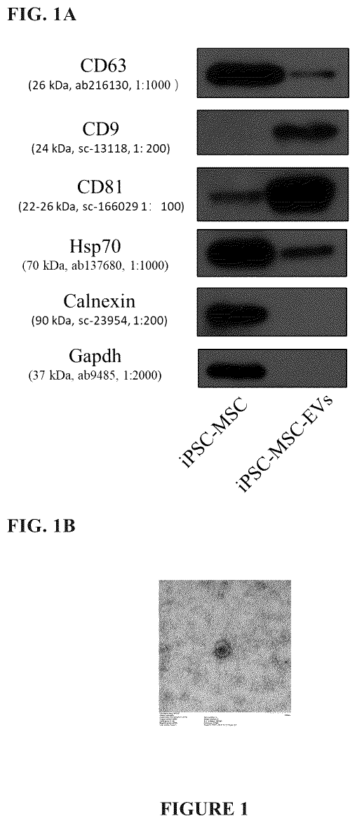

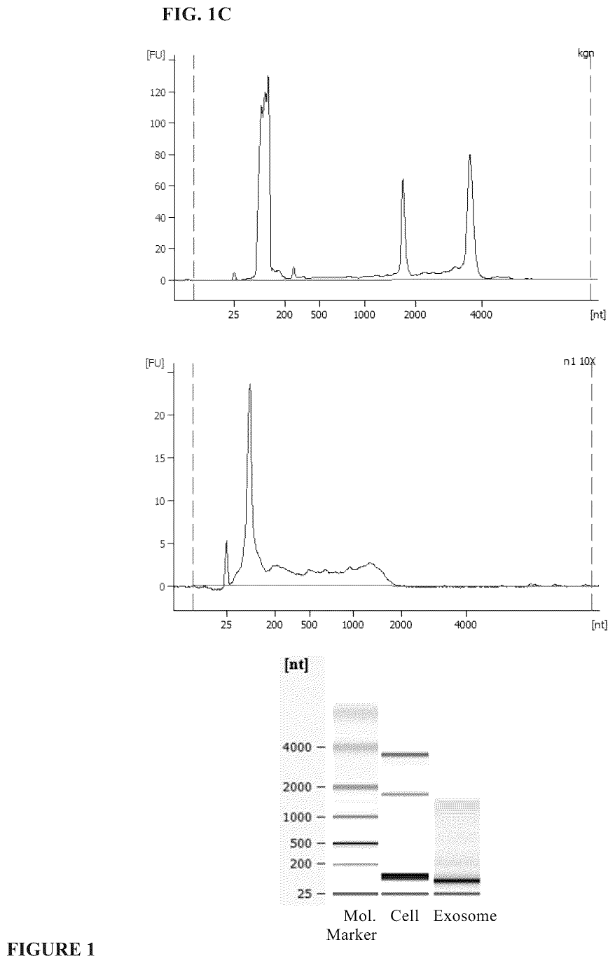

FIG. 1 shows the results of identification of exosomes obtained from human induced pluripotent stem cell-derived mesenchymal stem cells (iPSC-MSCs-EVs).

As shown in FIG. 1, iPSC-MSCs-EVs were characterized by immunoblotting, transmission electron microscopy (TEM), and RNA distribution analysis, respectively. Immunoblot (FIG. 1A) showed that the isolated exosomes were positive for the exosome surface markers CD63, CD9, CD81, and Hsp70, but negative for the exosome-negative marker Calnexin. By TEM (FIG. 1B), iPSC-MSCs-EVs showed a typical cup-shaped exosome structure with a diameter of about 40-150 nm. In addition, the distribution of RNA extracted from iPSC-MSCs-EVs was detected by Agilent 2100 Bioanalyzer (FIG. 1C), and the results showed that the peak of RNA was concentrated between 20-200 nt, which was significantly different with the RNA size distribution of cells.

The above identification characteristics indicated that exosomes were extracted from the supernatant of iPSC-MSCs cells.

Example 2 In vitro experiments show that iPSC-MSCs-EV treatment can regulate the ILK signaling pathway in granulosa cells.

By preparing granulosa cells with chemotherapy damage and co-culturing the injured granulosa cells with exosomes, the changes of the ILK signaling pathway therein were observed.

Granulosa cells from 20- to 23-day-old mice were isolated and divided into control group, cyclophosphamide (CTX)-induced apoptosis group, and cyclophosphamide-exosomes (CTX-EVs) co-treatment group. Among them, CTX was added to the cell culture medium at a concentration of 2 mg/ml, and exosomes (iPSC-MSCs-EVs) were added to the cell culture medium at a concentration of 20 μg/ml. The concentration of exosomes was determined using the Pierce™ BCA Protein Assay Kit, which is a total protein measurement kit. Incubated for 24 hours before the next step of detection was performed.

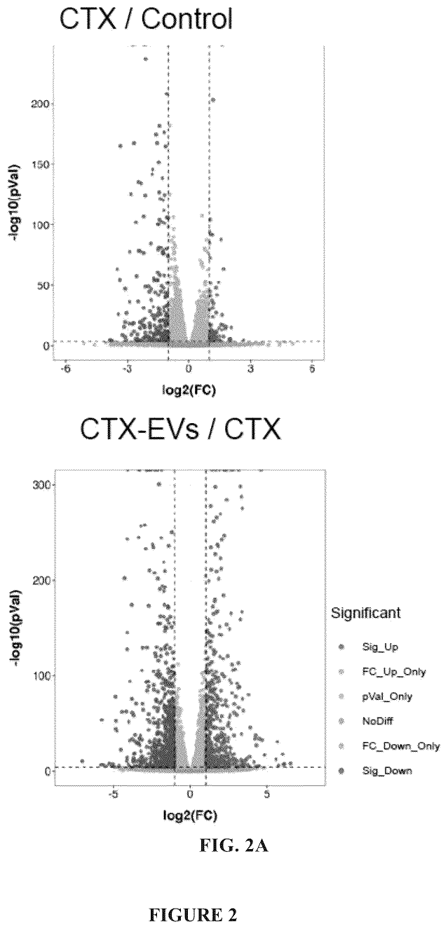

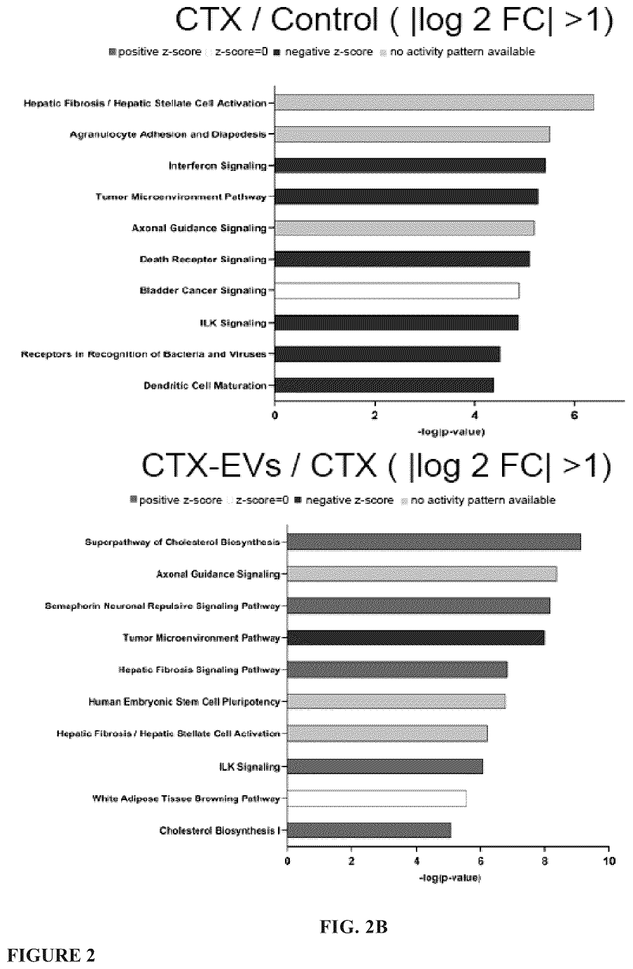

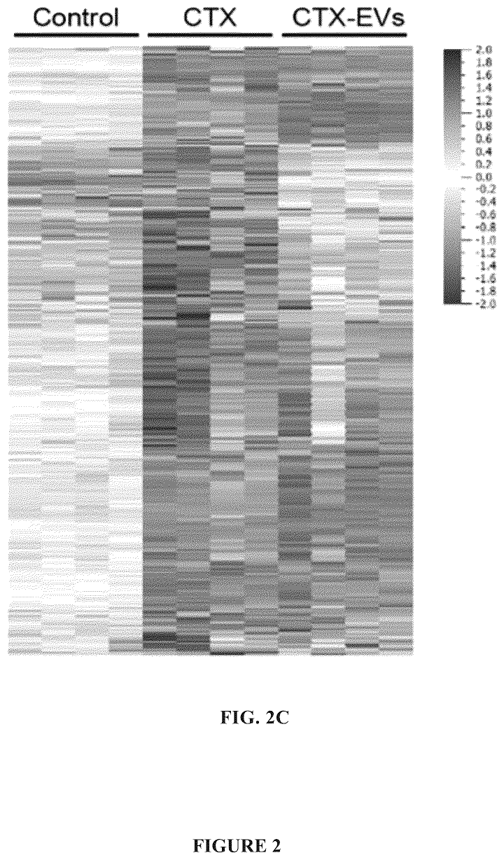

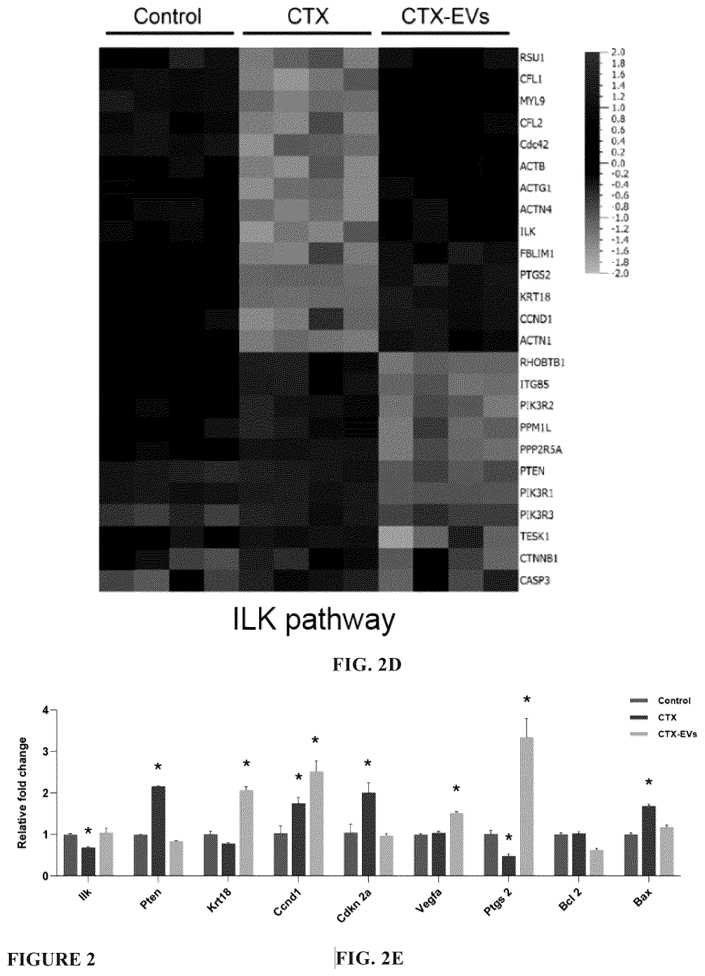

After the granulosa cells of the above experimental group were treated in vitro, RNA was extracted with Trizol, and transcriptome RNA sequencing was performed using the BGISEQ-500 system (BGI, Shenzhen, China). Sequencing results revealed that there were 1451 differentially expressed genes (DEGs) in the CTX treatment group compared with the CTX-EVs group (FIG. 2A). Carried out KEGG (Kyoto Encyclopedia of Genes and Genomes), and it was found that these differentially expressed genes were mainly related to the pathways of cell signal transfer and cell metabolism change; IPA software analysis showed that the ILK signaling pathway was inhibited in the CTX treatment group, while in the CTX-EV group was reactivated (FIG. 2B).

Use Qlucore software for further gene screening, select the 1000 genes with the most difference among the genes with expression change fold greater than 1.5 and p value less than 1.5 e (FIG. 2C), and then use IPA software to analyze these genes. The results show that the ILK signaling pathway is one of the most significant pathways that change. The heat map analysis of differential genes related to ILK signaling pathway further confirmed that compared with the control group, these differentially expressed genes were down-regulated in the CTX-treated group and up-regulated in the CTX-EV group (FIG. 2D).

Further analysis of ILK-related genes (including Ilk, Pten, Krt 18, Cond 1, Cdkn 2a, Vegfa, Ptgs2) by QPCR and confirmed the above changes.
ILK: Forward, 5′-GAACGACCTCAATCAGGGGG-3′; Reverse, 5′-CATTAATCCGTGCTCCACGC-3′;
Bcl2: forward, 5′-GAACTGGGGGAGGATTGTGG-3′; reverse, 5′-GCATGCTGGGGCCATATAGT-3′;
Bax: forward, 5′-TGAAGACAGGGGCCTTTTTG-3′; reverse, 5′-AATTCGCCGGAGACACTCG-3′;
Krt18: forward, 5′-ACCACCAAGTCTGCCGAAAT-3′; reverse, 5′-CCGAGGCTGTTTCTCCAAGTT-3′;
Ccnd1: forward, 5′-CAACTTCCTCTCCTGCTACCG-3′; reverse, 5′-GATGGAGGGGGTCCTTGTTTAG-3′;
Vegfa: forward, 5′-GCACATAGAGAGAATGAGCTTCC-3′; reverse, 5′-CTCCGCTCTGAACAAGGCT-3′;
Ptgs2: forward, 5′-CATCCCCTTCCTGCGAAGTT-3′; reverse, 5′-CATGGGAGTTGGGCAGTCAT-3′;
Cdkn2a: forward, 5′-CGCTTCTCACCTCGCTTGT-3′; reverse, 5′-AGTGACCAAGAACCTGCGAC-3′;
Pten: forward, 5′-TGGATTCGACTTAGACTTGACCT-3′; reverse, 5′-GCGGTGTCATAATGTCTCTCAG-3′;
Gapdh-: forward, 5′-GAGAGTGTTTCCTCGTCCCG-3′; reverse, 5′-ACTGTGCCGTTGAATTTGCC-3′.

Rat-Gapdh was used as an internal reference for normalization. Relative mRNA expression levels were determined using the 2-ΔΔCt method.

The results are shown in FIG. 2E. Compared with the control group, Ptgs2 (promoting the survival of cumulus granulosa cells during the expansion process) was down-regulated in the CTX treatment group, but was significantly up-regulated in the CTX-EV treatment group (p<0.05). The expression of apoptosis gene Bax was significantly increased in CTX group (p<0.05), but was significantly decreased in CTX-EV group (p<0.05). The anti-apoptotic gene Bcl2 was not significantly different among the groups. High-throughput miRNA sequencing revealed that 9 of the top 50 microRNAs (miRNAs) expressed in iPSC-MSC-EVs targeted PTEN, a lipid phosphatase that negatively regulates ILK activation. These results showed that iPSC-MSC EVs protected granulosa cells from apoptosis and maintained normal function by modulating the ILK signaling pathway, such as reversing the CTX-induced downregulation of the ILK pathway in granulosa cells by transferring functional miRNAs.

Example 3 iPSC-MSCs-EVs reverse PTEN/ILK/AKT pathway down-regulation in vivo or in vitro.

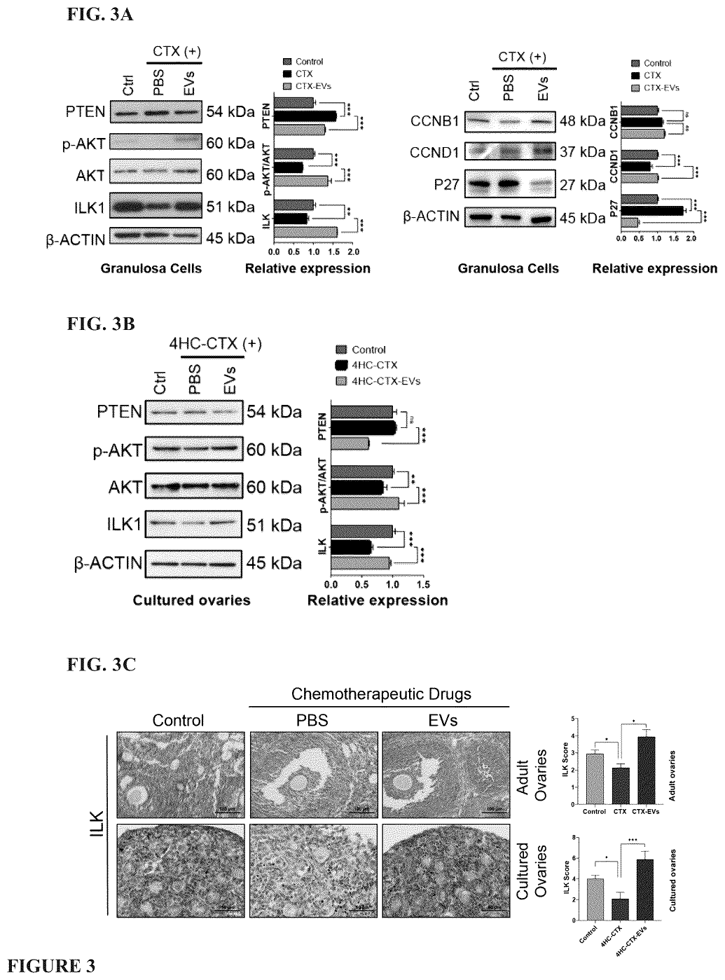

The specific expression of ILK signaling pathway-related proteins was further analyzed, and the protein of granulosa cells in the above experimental group was detected by immunoblotting. As shown in the results (FIG. 3A), the expression of ILK in the CTX group was lower than that in the control group, and the expression of ILK in the CTX-EV group was significantly increased. The expression of PTEN was significantly upregulated after CTX treatment, but was suppressed after adding iPSC-MSCs-EVs. The expression of ILK decreased significantly after CTX treatment, but increased significantly after adding iPSC-MSCs-EVs. The p-AKT/AKT ratio was significantly decreased after CTX treatment, but this decrease was significantly suppressed by the addition of iPSC-MSC-EVs. This means that CTX treatment inhibits the ILK/AKT pathway by upregulating the expression of PTEN, but iPSC-MSCs-EVs treatment can reverse this effect.

Since the ILK pathway is also involved in the regulation of the G1/S/G2 phase of the cell cycle, the cell cycle-related proteins Ccnb1, Ccnd1, and P27 were also checked in the experiment. The results showed that the expression of P27 was significantly increased after the addition of chemotherapeutic drugs, but decreased after the addition of iPSC-MSCs-EVs. The expression of Ccnd1 decreased significantly after the chemotherapeutic reagent was added, but increased significantly when iPSC-MSCs-EVs were added. There was no significant change in Ccnb1 (FIG. 3B). Western Blot was used to detect the expression changes of the ilk pathway in the ovaries cultured in vitro, and the results showed that the expression of PTEN could be significantly inhibited after iPSC-MSCs-EVs were added. The expression of IIK decreased significantly after adding chemotherapeutic reagent, but increased significantly after adding iPSC-MSCs-EVs. The p-AKT/AKT ratio decreased significantly after the addition of chemotherapeutics, but the addition of iPSC-MSCs-EVs significantly suppressed this decrease (FIG. 3C).

The expression of ILK in ovaries was detected by IHC staining of adult mouse ovaries and ovaries cultured in vitro. The results showed that in adult mice, the expression of ILK decreased after chemotherapy treatment but increased after iPSC-MSCs-EVs transplantation. The results of ovaries cultured in vitro were consistent with those of adult mice (FIG. 3D). These results showed that iPSC-MSCs-EVs reversed the CTX-induced downregulation of PTEN/ILK/AKT pathway in vitro and in vivo.

Example 4 In vitro experiments prove that iPSC-MSCs-EVs regulate the ILK signaling pathway to inhibit the apoptosis of granulosa cells

Granulosa cells (GCs) from mice of 20 to 23 days old were isolated and divided into a control group, a cyclophosphamide (CTX)-induced apoptosis group, and a co-treatment group of CTX and iPSC-MSCs-EVs.

CTX was added to the cell culture medium at a concentration of 4 mg/ml. iPSC-MSCs-EVs were added to the cell culture medium at a concentration of 2 μg/ml, 20 μg/ml or 100 μg/ml. After 24 hours of incubation, the next step of detection was performed.

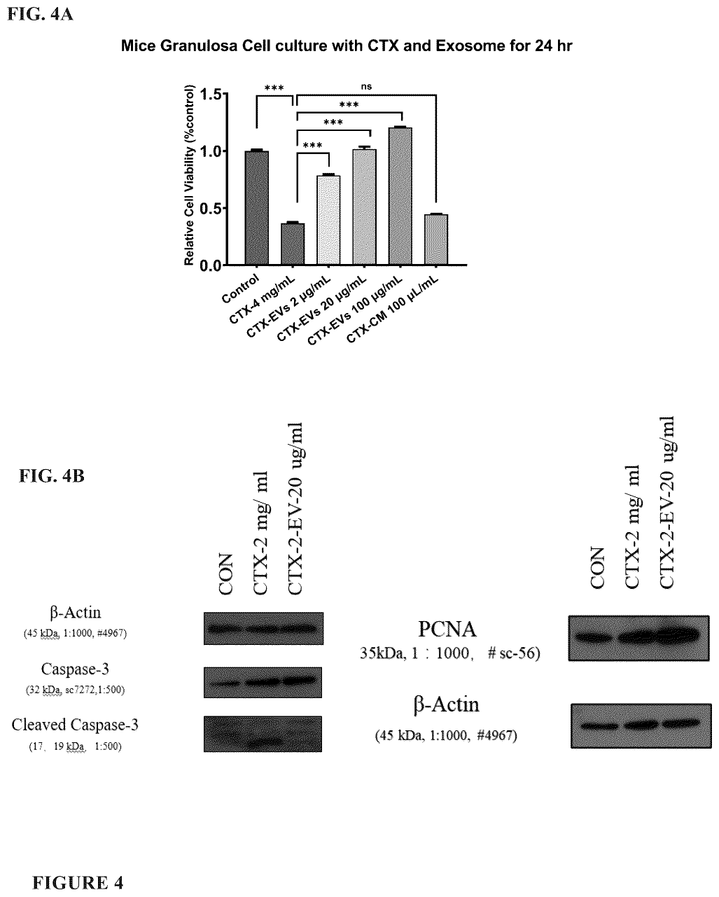

FIG. 4 is a graph showing the results of iPSC-MSCs-EVs treating CTX-induced apoptotic granulosa cells. By MTS assay, the results showed that iPSC-MSCs-EVs could greatly increase the cell survival rate in a dose-dependent manner compared with the CTX-treated group (FIG. 4A). Western blot results (FIG. 4B) showed CTX significantly increased the expression of apoptosis marker Cleaved Caspase 3 in granulosa cells, while iPSC-MSCs-EVs treatment significantly inhibited chemotherapy-induced apoptosis of granulosa cells, including reducing the expression of Cleaved Caspase 3, and increasing the expression of cell proliferation marker PCNA in granulosa cells.

The results showed that iPSC-MSCs-EVs treatment could inhibit the apoptosis of granulosa cells induced by CTX and promote their proliferation by regulating the ILK signaling pathway.

Example 5 Ovary culture experiments in vitro prove that iPSC-MSCs-EVs regulate ILK signaling pathway to protect follicle development and inhibit cell apoptosis.

The ovaries of 2.5-day-old mice were dissected and divided into control group, chemotherapy group treated with 10 μM 4-hydroxycyclophosphamide (4HC-CTX), or co-processing group treated with 10 μM 4HC-CTX and 100 μg/ml iPSC-MSC-EVs. Incubate for 72 hours.

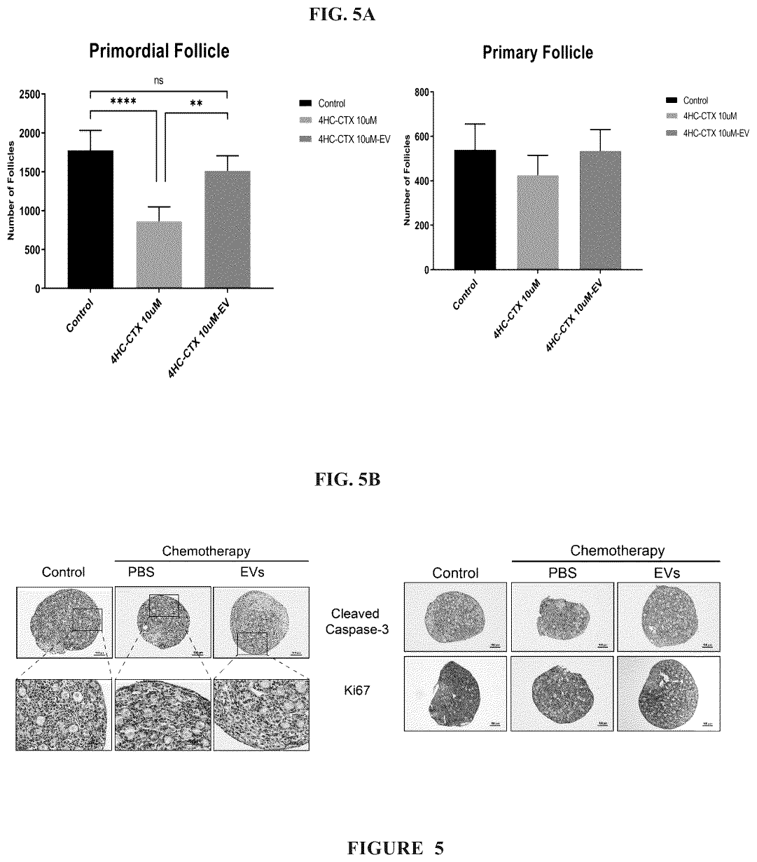

FIG. 5 is a graph showing the results of co-culture of iPSC-MSCs-EVs and 4HC-CTX in ovaries.

The follicle count results (FIG. 5A) showed that the primordial follicles of ovaries cultured with 4HC-CTX were significantly less than those of the control group. But in the ovaries co-treated with 4HC-CTX and EVs, the number of primordial follicles was significantly increased compared with the 4HC-CTX group. The results of immunohistochemical experiments (FIG. 5B) showed that compared with the 4HC-CTX group, iPSC-MSC-EVs decreased the expression of Cleaved Caspase 3 and increased the expression of PCNA.

The results showed that iPSC-MSC-EVs could inhibit the apoptosis induced by CTX and promote the recovery of ovary.

Example 6 In vivo experiments prove that hiPSC-MSCs-EVs have a protective effect on ovarian cell apoptosis in mice.

The 28-day-old mice were divided into treatment groups, including control group, CTX group and CTX-EV group. For the mice in the CTX group, CTX (120 mg/ml) was injected intraperitoneally once a week, twice in total, and 200 μl of normal saline was injected into the tail vein six times on the 1st, 3rd, 5th, 10th, 12th, and 14th day, respectively. For the mice in the CTX-EV group, CTX (120 mg/ml) was injected intraperitoneally once a week, twice in total, and 200 μg EV were inject into the tail vein six times on the 1st, 3rd, 5th, 10th, 12th, and 14th day, respectively. For mice in the control group, the same dose of saline was injected.

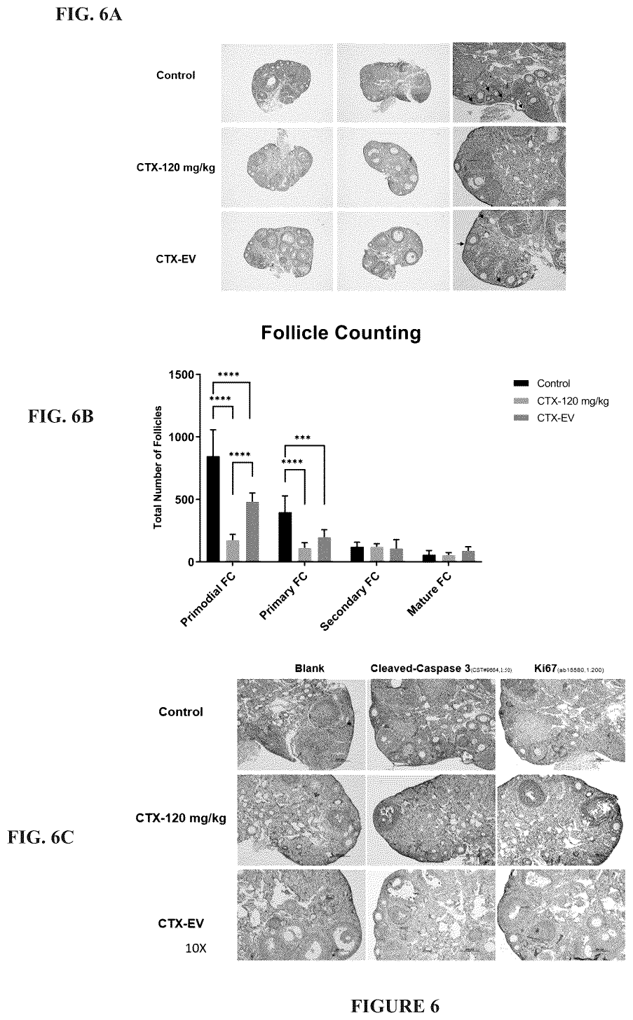

FIG. 6 is a graph showing the effect on the ovaries of mice treated with CTX and co-cultured with iPSC-MSCs-EVs.

As shown in FIG. 6A, in the control group, a large number of normal follicles at different stages could be observed in the ovaries. The number of primordial follicles and primary follicles was significantly decreased in the CTX group mice, but in the CTX-EV group, the number of primordial follicles was significantly increased compared with the CTX group, and the number of primary follicles was also significantly increased. Follicle count results (FIG. 6B) further demonstrated a statistically significant reduction in the number of primordial follicles and primary follicles in mice in the CTX group in comparison. But in the CTX-EV group, the number of primordial follicles was significantly increased compared with the CTX group, and the number of primary follicles was also significantly increased. For secondary follicles and mature follicles, there was no significant difference among the three groups.

The results of immunohistochemical experiments (FIG. 6C) also proved that iPSC-MSC-EVs could reduce the expression of apoptosis marker Cleaved Caspase 3 and increase the expression of cell proliferation marker Ki67.

The results showed that iPSC-MSC-EVs could regulate the ILK signaling pathway to inhibit chemotherapy-induced apoptosis and promote ovarian recovery.

The above is an explanation of the present invention, and it should not be regarded as a limitation of the present invention. The practice of the present invention will employ, unless otherwise indicated, conventional techniques of organic chemistry, polymer chemistry, biotechnology and the like, and it will be apparent that the invention can be carried out otherwise than as specifically described in the foregoing specification and examples. Other aspects and modifications within the scope of the invention will be apparent to those skilled in the art to which the invention pertains. Many modifications and variations are possible based on the teachings of the present invention and are therefore within the scope of the present invention.

In the present invention, “about” means ±10%, preferably ±5%, more preferably ±2%, such as #1%, ±0.5% or ±0.1%.

1.-20. (canceled)

21. A method for regulating the ILK signaling pathway in a cell, comprising administering exosomes from induced pluripotent stem cell-derived mesenchymal stem cells to the cell.

22. The method according to claim 21, wherein the mesenchymal stem cells are cells that have undergone 1-10 passages.

23. The method according to claim 21, which is a method for regulating the ILK signaling pathway in cultured cells in vitro, comprising adding exosomes from induced pluripotent stem cell-derived mesenchymal stem cells to the cell culture medium.

24. The method according to claim 23, wherein the amount of exosomes added is about 1-500 μg/ml.

25. The method according to claim 21, wherein the cell is a cell with abnormal ILK pathway.

26. The method according to claim 21, wherein the ILK pathway activity of the cell is restored by upregulating the ILK activity of the cell.

27. A method for treating diseases related to ILK signaling pathway, comprising administering exosomes from induced pluripotent stem cell-derived mesenchymal stem cells to patients.

28. The method according to claim 27, wherein the mesenchymal stem cells are cells that have undergone 1-10 passages.

29. The method according to claim 27, wherein administering about 1-500 μg exosomes to the patient.

30. The method according to claim 27, wherein the exosomes are administered to the patient for multiple times.

31. The method according to claim 30, wherein the exosomes are administered to the patient at intervals of about 1 day to 7 days.

32. The method according to claim 31, wherein each administration dosage is about 1-500 μg.

33. The method according to claim 27, wherein the ILK signaling pathway-related diseases are abnormal or pathological angiogenesis-related diseases, metabolic disorders, inflammatory diseases or ovarian-related reproductive disorders.

34. The method according to claim 22, wherein the mesenchymal stem cells are cells that have undergone 3-7 passages.

35. The method according to claim 24, wherein the amount of exosomes added is about 10-200 μg/ml.

36. The method according to claim 25, wherein the cell is a cell with down-regulated ILK activity.

37. The method according to claim 28, wherein the mesenchymal stem cells are cells that have undergone 3-7 passages.

38. The method according to claim 29, wherein administering about 10-200 μg exosomes to the patient.

39. The method according to claim 31, wherein the exosomes are administered to the patient at intervals of about 2 to 5 days.

40. The method according to claim 32, wherein each administration dosage is about 10-100 μg.
